(19) United States
(12) Patent Application Publication
(10) Pub. No.: US 2003/0162255 A1
(43) Pub. Date: Aug. 28, 2003

(54) Type-C lectins and methods of use

(76) Inventors: Jeffery Fairman, Mountain View, CA (US); Choong-Chin Liew, Boston, MA (US)

Correspondence Address: ROBERT D. FISH; RUTAN & TUCKER, LLP, P.O. BOX 1950, 611 ANTON BLVD., 14TH FLOOR, COSTA MESA, CA, 92628-1950, US

(21) Appl. No.: 10/349,437

(22) Filed: Jan. 21, 2003

Related U.S. Application Data
(60) Provisional application No. 60/351,501, filed on Jan. 23, 2002.

(51) Int. Cl. A61K 48/00; C12P 21/02; C12N 5/06; C07K 14/47

(52) U.S. Cl. 435/69.1; 435/320.1; 435/325; 530/395; 536/23.5; 514/44

(57) ABSTRACT

The nucleotide and corresponding protein sequence of a gene belonging to the family of type-C lectins are disclosed. In particularly contemplated aspects, native and recombinant full length and mutant variants of the novel type-C lectin are employed for use in diagnostic and therapeutic applications

This application claims the benefit of U.S. provisional application No. 60/351,501, which was filed Jan. 23, 2002, and which is incorporated herein by reference.

### FIELD OF THE INVENTION

The field of the invention is recombinant and non-recombinant nucleic acids and their corresponding polypeptides, especially relating to lectins.

### BACKGROUND OF THE INVENTION

The group of lectins encompasses a relatively large and diverse population of molecules that may be broadly characterized as proteins, which specifically bind or crosslink carbohydrates, and which may further have catalytic activity (e.g., RNA-N-glycosidase activity of ricin). Depending on their particular origin and/or biochemical activity, lectins can be classified into legume lectins (e.g., ConA from Concanavalia ensiformis), invertebrate lectins (e.g., lectin from ascidian Didemnum ternatanum), galectins (typically binding galactose with relatively high specificity), annexins (typically binding selected lipids or glycosaminoglycans), and soluble and transmembrane C-type lectins (generally requiring Ca²⁺ for binding and/or structural integrity).

Both soluble and transmembrane C-type lectins have recently gained considerable attention due to their potential usefulness in various diagnostic and therapeutic applications. For example, Clark et al. reported potential use of collectins (soluble C-type lectins) in treatment of infectious agents in the lung [Clark, H. W., Reid, K. B., and Sim, R. B.: Collectins and innate immunity in the lung; Microbes Infect 2000; 2(3):273-8]. Although collectins exhibit various desirable properties (e.g., generally soluble in a biological fluid, relatively high binding specificity towards their target moiety), recombinant and/or large-scale production of collectins tends to be difficult due to the particular biochemical structure of the collectins (N-terminal collagen domain coupled to a CRD (carbohydrate recognition/binding domain).

In another example, E-selectin (a transmembrane C-type lectin) and its plasma soluble homologue can be employed as a marker for inflammatory processes, especially for myocardial infarction as reported by Suefuji et al. [Suefuji, H., et al.: Increased plasma level of soluble E-selectin in acute myocardial infarction; Am Heart J 2000; 140(2):243-8]. Suefuji's test exhibits a relatively conclusive correlation between the occurrence of a myocardial infarction and elevated sE-selectin. However, events in patients with enhanced endothelial cell activation due to causes other than acute myocardial infarction (AMI) tend to reduce the prognostic value for AMI of this test.

In a further example, McAbee et al. report that the asialoglycoprotein receptor (a transmembrane type-11 receptor lectin) on hepatocytes can be targeted with lactoferrin [McAbee, D. D., et al.: Lactoferrin binding to the rat asialoglycoprotein receptor requires the receptor's lectin properties; Biochem J 2000; 348, Pt 1:113-7], thereby potentially opening an avenue for hepatospecific targeting of drugs. Although McAbee's system is conceptually relatively specific towards its target, it is generally necessary that the asialoglycoprotein receptor be expressed in sufficient quantities on the target cells, which may not always be the case.

In yet another example, Lasky et al. describe in U.S. Pat. No. 6,117,977 (Sep. 12, 2000), which is incorporated by reference herein, an endocytic type-C lectin (a transmembrane lectin with tandem CRDs) as being useful as an expression marker or therapeutic agent that competes with normal binding of native proteins to their ligands. Lasky et al. add another member to the continuously expanding class of type-C lectins, however, the physiological role and biochemical significance of Lasky's lectin appears not to be fully understood.

Although various lectins and particularly type-C lectins are known in the art, all or almost all of them appear to be limited to a particular physiological environment and/or biochemical function. Thus, there is a continuous need to provide compositions and methods for novel type-C lectins.

### SUMMARY OF THE INVENTION

The present invention is directed to novel type-C lectin nucleic acid and polypeptide sequences. Contemplated molecules particularly include type-C lectins that bind a molecule comprising a carbohydrate residue and have a general structure of a transmembrane (type II receptor) type-C lectin. The novel type-C lectin has a nucleotide sequence according to SEQ ID NO:1, and a corresponding polypeptide sequence according to SEQ ID NO:4.

In one aspect of the inventive subject matter, the nucleotide sequence comprises a signal peptide domain which partially overlaps with a transmembrane domain, and a carbohydrate binding domain, and it is contemplated that at least one of those domains may be deleted at least in part. The transmembrane domain and carbohydrate binding domain have the nucleotide sequences according to SEQ ID NO:2 and 3, respectively.

In another aspect of the inventive subject matter, the type-C lectin nucleic acid of fragments thereof are operationally coupled to a control sequence that is recognized by a host cell transformed with such nucleic acids. Contemplated host cells include prokaryotic and eukaryotic host cells, and particularly contemplated host cells are E. coli, Saccharomyces cerevisiae, SF6 cells, and where the cells are located in-vivo, especially contemplated host cells are part of a mammal.

In a further aspect of the inventive subject matter, the polypeptides according to SEQ ID. NOS: 4-6 or a fragment thereof are coupled to a non-type-C lectin polypeptide, preferably at least a portion of an immunoglobulin, and more preferably a constant part of the heavy chain of an immunoglobulin G.

In a still further aspect of the inventive subject matter, a type-C lectin can be employed to regulate the uptake of a drug into a cell by correlating the uptake of the drug with the presence of a type-C lectin on a surface of the cell. The cells are transformed with a nucleic acid encoding at least a portion of the type-C lectin, and the nucleic acid is transcribed in the cell to a transcript, which either reduces expression of cellular type-C lectin via anti-sense hybridization, or increases type-C expression via translation of the transcript.

Various objects, features, aspects and advantages of the present invention will become more apparent from the following detailed description of preferred embodiments of the invention, along with the accompanying drawing.

### BRIEF DESCRIPTION OF THE DRAWING

FIG. 1 is a schematic view of a nucleic acid comprising a type-C lectin according to SEQ ID No. 1.

FIG. 2 is a schematic view of vector comprising a type-C lectin according to SEQ ID No. 1.

FIG. 3 is a schematic view of a cell comprising the vector of FIG. 2.

### DETAILED DESCRIPTION

The novel type-C lectin nucleic acid having the nucleotide sequence of SEQ ID NO: 1 has been isolated from a cDNA library obtained from human umbilical cord blood using standard random priming methods. The isolated nucleic acid has been sequenced in an ABI 377 and identified on the nucleic acid level as a type-C lectin by its pronounced homology of 50% identity with over 215 amino acids of a mouse macrophage C-type lectin employing the “PBLAST” sequence comparison program. Structural analysis by “SMART” (Simple Modular Architecture Research Tool) (Schultz, J., Milpetz, F., Bork, P., and Ponting, C. P. (1998) SMART, a simple modular architecture research tool: Identification of signaling domains Proc. Natl. Acad. Sci. USA 95, 5857-5864) and “PFAM” (Protein families database) both indicated the presence of C-type lectin domains.

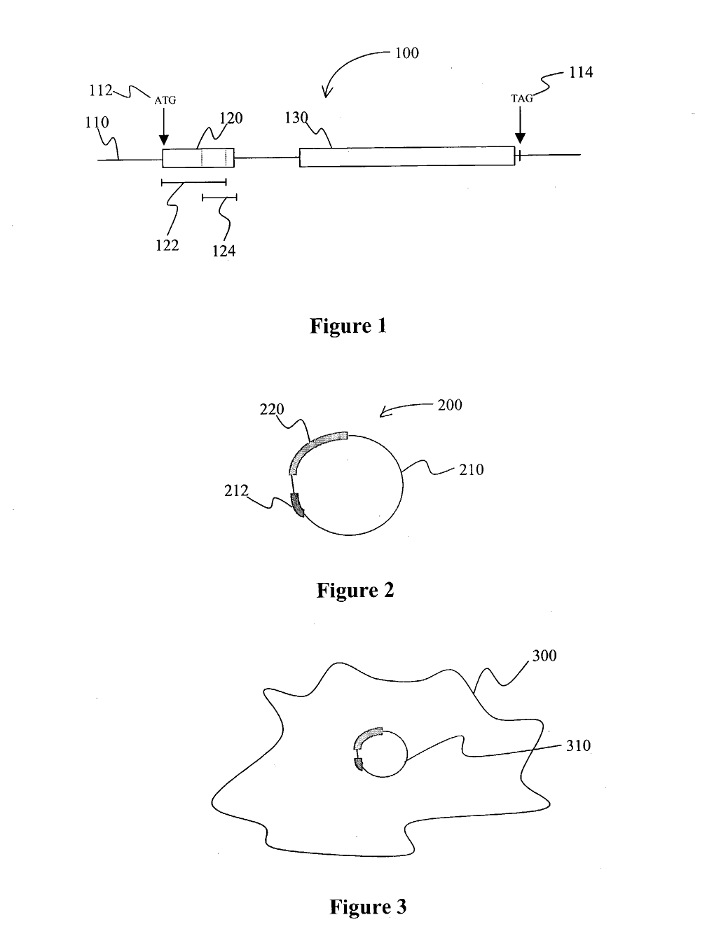

The general organization of a nucleic acid comprising a sequence according to SEQ ID NO1 is depicted in FIG. 1, in which the nucleic acid 100 has 5′ non-coding/regulatory region 110. The coding region for the type-C lectin of the nucleic acid 100 starts at the start codon ATG 112, which defines the 5′-end of the structural/functional element 120 that includes a leader sequence 122 and an in-frame transmembrane domain 124. A second structural/functional element 130 includes the carbohydrate binding domain and is followed by the stop codon 114. As used herein, the term “carbohydrate recognition domain” and “carbohydrate binding domain” are used interchangeably and refer to a domain in a molecule that binds a carbohydrate with an affinity of K_{D}≦10⁻³/mol.

With respect to the source from which the novel type-C lectin can be isolated, it should be appreciated that numerous sources other than a cDNA library from umbilical cord blood are also suitable, and appropriate sources include nucleic acid libraries such as human partial or full length cDNA libraries, genomic DNA libraries in various forms (e.g., phage libraries, cosmid libraries, YAC libraries, etc.), and expression libraries. Contemplated libraries may thereby be organ-, or tissue specific, specific to a particular developmental state of an organ or organism, and may be derived from various enrichment procedures, especially including subtractive hybridization procedures. There are numerous library construction techniques and protocols known in the art, and all of them are contemplated suitable fro use in conjunction with the teachings presented herein. An exemplary collection of protocols can be found in cDNA Library Protocols by Ian G. Cowell (Editor), Caroline A. Austin (Editor), 1997, Humana Press; ISBN: 089603383X and Molecular Cloning. A Laboratory Manual by T. Maniatis et al., Cold Spring Harbor Laboratory Press; ISBN: 0879693096.

Alternatively, it is contemplated that the type-C lectin may be isolated from crude or purified DNA and/or RNA preparations, which may be derived from a cell or tissue culture, a biopsy specimen, an organ, or other suitable source such as commercially available DNA and/or RNA sources. It should further be appreciated that the format of alternative DNA and/or RNA preparations need not be limited to a lyophilized or dissolved preparation, but may also include immobilized nucleic acids, and particularly contemplated immobilized forms include DNA and/or RNA preparations on a filter (e.g., nitrocellulose or other support), on a glass support, and on a gene chip. On the other hand, where appropriate, contemplated type-C lectins may also be directly isolated from one or more cells, a tissue sample, organ or entire organism. Numerous DNA and RNA procedures and test kits are known in the art, and all of which are contemplated suitable for use herein. Exemplary protocols may be found in Molecular Cloning: A Laboratory Manual by T. Maniatis et al. (supra), and kits for DNA and RNA isolation are available from QIAGEN Inc. USA, 28159 Avenue Stanford, Valencia, Calif. 91355.

While it is generally preferred that the type-C lectin is isolated from a mammal, most preferably a human, alternative organisms are also contemplated, including vertebrates and non-vertebrates.

Depending on the nucleic acid source, it should be appreciated that contemplated type-C lectin may be isolated employing various procedures, and particularly suitable procedures include library screening with labeled and/or unlabeled nucleic acid probes (typically less than 500 bases) or labeled and/or unlabeled antibodies, PCR methods, and selective hybridization procedures in a column or otherwise chromatographic format. For example, where the source material comprises a full-length CDNA library, screening can be done with a radiolabeled (e.g., ³²P or ³³P) oligonucleotide. On the other hand, where antibodies are available that specifically bind the type-C lectin, screening may be performed in an expression library following an immunoscreening protocol. If no libraries are available, PCR methods with primers targeted against at least one strand within the type-C gene are contemplated, and particularly preferred PCR protocols include RT-PCR, RACE-PCR, inverse PCR, and nested primer PCR. There are many PCR methods for isolation of genes and/or gene fragments known in the art, and an exemplary collection of suitable protocols can be found in PCR Applications: Protocols for Functional Genomics by Michael A. Innis (Editor), David H. Gelfand (Editor), John J. Sninsky (Editor), John J. Sninksy, John Sninsky; Academic Press; ISBN: 0123721865.

In a particularly preferred aspect, the isolation of contemplated type-C genes may also be at least partially performed in silico, employing a database of known sequences against which a query with one or more known nucleic acid sequences encoding known type-C lectins is run. See e.g., Computational Methods in Molecular Biology (New Comprehensive Biochemistry, Vol 32) by Steven L. Salzberg (Editor), David B. Searls (Editor), and Simon Kasif (Editor). Elsevier Science; ISBN: 0444502041. Unknown sequences with homology of more than 50%, more preferably more than 70%, and most preferably more than 85% are then candidates for further structural and/or functional analysis, which may include bio-informatics analysis to determine similarity to known crystal structures, folding patterns, or other consensus motifs. Further analysis of candidate sequences include physical isolation of the sequence and expression followed by functional analysis to confirm the identity of the candidate sequence as a type-C lectin.

With respect to establishing the exact nucleic acid sequence, it is contemplated that numerous methods other than automated sequencing on an ABI 377 are also appropriate, and particularly suitable sequencing methods include manual and automated chain termination methods (e.g., Sanger, F. and Coulson, A. R.; A rapid method for determining sequences in DNA by primed synthesis with DNA polymerase; J. Mol. Biol. 1975;94(3):441-8), thermal cycle sequencing (e.g., PCR Applications. Protocols for Functional Genomics, supra), and chemical degradation methods (e.g., Maxam, A. M., and Gilbert, W. 1980. Sequencing end-labeled DNA with base-specific chemical cleavages. Methods in Enzymol. 65: 499-560).

In a further aspect of the inventive subject matter, it is contemplated that the type-C lectin is expressed in a host cell, wherein the expression may be homologous or heterologous. The term “homologous expression” as used herein means that the expressed gene is isolated from the same species and genus to which the host cell belongs. In contrast, the term “heterologous expression” as used herein means that the expressed gene is isolated from a species and genus other than the species and genus to which the host cell belongs. There are numerous methods of gene expression known in the art, and depending on the particular construct and/or desirable biochemical properties of genes a particular expression strategy can be employed. Suitable exemplary strategies and protocols can be found among other sources in Gene Expression Systems: Using Nature for the Art of Expression by Joseph M. Fernandez (Editor), Joe Fernandez (Editor), James P. Hoeffler (Editor); Academic Press; ISBN: 0122538404, or in Gene Transfer and Expression: A Laboratory Manual by Michael Kriegler; Oxford Univ Press; ISBN: 0716770040.

It is generally contemplated that the nucleic acid encoding contemplated type-C lectins are (1) part of a vector or linear piece of nucleic acid, and (2) are operationally coupled to a control sequence that is recognized by the host cell transformed with the vector or linear piece of nucleic acid. The term “vector” as used herein refers to a DNA or RNA molecule (typically double stranded) that may replicate independently from the genome of the host cell, or that may replicate after integration into the genome of the host cell together with the host cell. The term “operationally coupled to a control sequence” as used herein refers to placement of the nucleic acid to be expressed in a sequence context that allows regulated or unregulated transcription of the nucleic acid. Contemplated control sequences include promotor elements, polymerase binding sites, hormone responsive elements, ribosome binding sites, operator elements for cis- and transacting molecules, enhancer elements, etc. An exemplary vector is depicted in FIG. 2, in which a vector 200 comprises a sequence portion 210 that effects replication independent from the host cell (e.g., ori), and that further includes a selection marker (e.g., ampicillin resistance). Sequence portion 212 comprises a control sequence (e.g., transcription initiation, ribosome binding site, etc.), which is operationally coupled to SEQ ID NO:1.

With respect to the host cell, it is generally contemplated that the expression in the host cell is performed in vitro, however, it is particularly contemplated that the expression may also be performed in vivo. Where prokaryotic expression systems are employed (e.g., to avoid glycosylation), it is generally contemplated that both gram-negative and gram-positive cells are suitable. Particularly contemplated gram-negative cells include various E. Coli strains such as 294, X776, or W3110, and especially contemplated gram-positive cells include various Bacillus species. Alternatively, expression may also be performed in eucaryotic microbes, including yeast strains and fungi, and particularly preferred microbes are Pichia pasteuris, Saccharomyces spec., and Kluyveromyces spec. An exemplary host cell 300 harboring the vector of FIG. 2 is depicted in FIG. 3.

Where it is particularly desirable to include glycosylation of a higher eukaryotic organism, multicellular host are especially contemplated. For example, SF-9 cells from Spodoptera frugiperda may be transformed with a recombinant baculovirus that harbors at least part of the type-C lectin nucleic acid. However, there are numerous alternative virus- and non-virus based eukaryotic animal and plant expression systems known in the art, and all of such known systems are contemplated suitable for use herein. In a particularly contemplated aspect, selected cells of an organism, preferably a mammal, are transformed with a nucleic acid encoding the contemplated novel type-C gene. Exemplary suitable models and protocols can be found in Gene Therapy Technologies, Applications and Regulations: From Laboratory to Clinic by Anthony Meager (Editor); John Wiley & Sons; ISBN: 0471967092, or in Nonviral Vectors for Gene Therapy by Leaf Huang (Editor), Mien-Chie Hung (Editor), Ernst Wagner (Editor); Academic Press; ISBN: 0123584655.

The choice of a suitable vector will predominantly depend on the type of host cell, available restriction sites within the vector and/or nucleic acid encoding for the novel type-C lectin, and it is generally contemplated that all commercial vectors or their modifications are appropriate. For example, suitable vectors may be obtained from ATCC (10801 University Boulevard, Manassas, Va. 20110-2209) or various alternative commercial sites (e.g., New England Biolabs, Inc., Boehringer Mannheim, etc.). Where integration of the recombinant DNA is particularly linear DNA carrying the type-C nucleic acid or fragment thereof may be employed to transform the host cell. Similarly, the choice of linear DNA will generally depend on the particular expression system, however it is contemplated that the type-C encoding nucleic acid is operationally coupled to a control sequence.

With respect to subcloning the nucleic acid into the appropriate vector or linear DNA, it is contemplated that all known cloning techniques are appropriate, and an exemplary collection of cloning techniques can be found in Molecular Cloning: A Laboratory Manual by T. Maniatis et al. (supra). While it is generally preferred that the entire coding sequence of the novel type-C gene is expressed in the native form, all mutant forms of the novel type-C gene are also contemplated. The term “native” as use herein refers to the sequence as originally isolated from its host organism. Particularly contemplated mutant forms include deletions, insertions, transitions and transversions.

For example, it is contemplated that a deletion may advantageously remove at least some of the cytoplasmic portion to render the mutant physiologically silent with respect to the inside of a cell. Alternatively, the transmembrane portion may be removed to render the mutant form soluble in plasma, and in still further contemplated aspects, the carbohydrate binding domain may be mutated (e.g., by insertion, deletion, single nucleotide exchange, etc.) to alter the binding behavior (with respect to the wild-type type-C lectin) of the mutant forms. Similarly, N-terminal or C-terminal portions may be removed to reduce molecular weight, or to improve expression or solubility of the recombinant mutant protein.

In another example, insertions to the sequence may be employed to add or replace one or more CRDs from the same type-C lectin or other lectins, or to add properties from entirely different molecules. Especially contemplated additions include nucleic acid sequences from non-type-C lectin polypeptides (i.e., any natural and/or synthetic polypeptide other than a type-C lectin) and especially include immunoglobulins, T-cell receptors, and catalytically active moieties, which may include entire enzymes or portions thereof. Among other moieties, particularly preferred catalytically active moieties are useful in generating a colorimetric, luminometric, or fluorometric quantifiable signal (e.g., peroxidases, luciferases), and non-catalytic moieties include fluorescent proteins (e.g., GFP, eGFP, BFP, etc.). Where it is preferred to radiolabel the recombinant mutant protein, poly-tyrosine portions may be included for radio-iodine labeling, however, alternative radiolabeling is also contemplated (e.g., via chelation, phosphorous-isotope incorporation, etc.).

In yet another example, one or more point mutations, which may be clustered, site-directed, or random may be introduced into the nucleic acid sequence coding for the novel type-C lectin. For example, where codon-usage of the host is biased towards one set of codons for one or more amino acids, and the native sequence utilizes preferentially another set of codons for the same amino acids, it is contemplated that silent point mutagenesis may be employed to assimilate the native sequence to the preference of the host cell. On the other hand, where a particular amino acid is involved in misfolding or aggregation of the recombinant type-C lectin, amino acid replacement with homologous or non-homologous amino acids are particularly contemplated. In still further contemplated site-directed mutagenesis strategies, changes are incorporated to ‘humanize’ the recombinant type-C lectin where the type-C lectin nucleic acid is isolated from an organism other than a human. In further examples of point-mutageneses, random changes may be introduced to create a library of mutants from which then potential candidates with particularly desirable properties may be selected (e.g., phage panning or other affinity-based selection strategies).

Therefore, it should be appreciated that not only sequences according to SEQ ID NO:1-3 are contemplated, but also nucleic acids with a sequence identity of greater than 75%, more preferably greater than 85%, and most preferably greater than 95%. It is contemplated that such mutant sequences will typically hybridize with the native novel type-C nucleic acid under stringent conditions. The term “stringent conditions” as used herein refers to low ionic strength and high temperature in the washing step after binding (e.g., 0.015 sodium chloride/0.0015 M sodium citrate/0.1% sodium dodecyl sulfate at 50° C.), or using a denaturing agent during hybridization (e.g., 50% (vol/vol) formamide in wash buffer). Further examples and protocols for hybridization under stringent conditions are described in Molecular Cloning. A Laboratory Manual by T. Maniatis et al. (supra).

In yet another especially contemplated aspect of the inventive subject matter, the recombinant type-C lectin polypeptide or fragments thereof (supra) may be covalently or noncovalently modified, and particularly preferred modifications include addition of a chromophore or chromogen, addition of an affinity label, addition of an radiolabel, addition of an enzymatic function, and addition of polyhydric polymers. For example, where the recombinant type-C lectin polypeptide is employed in a diagnostic test, suitable modifications may include halogenated indolyls (e.g., 5-Br-4-Cl-3-indolylacetyl) for colorimetric detection, NAD-analogs or fluorescein-type compounds for fluorometric detection, biotin labels for detection using avidin, chelators for binding of radioactive metals, or luciferase as a bioluminescence generator. In further particularly preferred aspects of the inventive subject matter, recombinant type-C lectin polypeptides or fragments thereof may also be complexed or covalently bound to a polymer or other substance that increases serum half-life time and/or reduces immunogenicity, and such polymers and substances particularly include polyhydric polymers (e.g., polythylene glycol).

With respect to additional polypeptide components, it is particularly contemplated that the recombinant type-C lectin polypeptide may be employed in the fabrication of immunoadhesins, which may be constructed by in-frame addition of suitable sequences to the recombinant type-C lectin nucleic acid, or by directly or indirectly covalently binding suitable polypeptide portions. Suitable embodiments of immunoadhesins are disclosed, for example, in U.S. Pat. No. 6,117,977, columns 22-26, which is incorporated by reference herein.

With respect to the binding specificity of contemplated sequences, it should be appreciated that a particular specificity will depend on the exact sequence of a particular type-C lectin. However, it is generally contemplated that peptides according to SEQ ID NOS 4 and 6 exhibit binding specificity consistent with the class of transmembrane C-type lectin (type II receptors) and it is further contemplated that such binding specificity is particularly directed towards carbohydrates, which preferably comprise modified and unmodified galactose.

Contemplated Uses of Recombinant Type-C Lectin Nucleic Acids and Polypeptides

(1) Therapeutic Uses

It is generally contemplated that the recombinant type-C lectin nucleic acids, polypeptides, or fragments thereof may be employed in a variety of therapeutic applications, and particularly contemplated applications include transformation of a host cell with a recombinant type-C lectin nucleic acid or fragments thereof to complement a lack of expression of the wild-type gene of the host cell, or to increase an already existing expression of the wild-type gene of the host cell. Alternatively, the recombinant type-C lectin nucleic acid or fragments thereof may be employed to reduce the expression of the wild-type gene in the host cell via an anti-sense transcript. For example, recombinant expression or transcription of the novel type-C lectin nucleic acid is thought to be especially advantageous, where the type-C lectin nucleic acid encodes an importer or import-facilitating protein. Consequently, it is contemplated that a method of regulating the uptake of a drug into a cell may have a step, in which the uptake of the drug is correlated with the presence of a type-C lectin on a surface of the cell. In a further step, the cell is transformed with a nucleic acid, wherein the nucleic acid encodes the type-C lectin, and wherein the nucleic acid is transcribed within the cell to produce a transcript, and in a subsequent step, the transcript alters at least indirectly the amount of the type-C lectin on the surface of the cell.

With respect to the step of transformation of a cell, it is contemplated that all viral and non-viral transformation methods are suitable for use in conjunction with the teachings presented herein, and exemplary methods and protocols are described in Gene Therapy Technologies, Applications and Regulations: From Laboratory to Clinic by Anthony Meager (Editor); John Wiley & Sons; ISBN: 0471967092, or in Nonviral Vectors for Gene Therapy by Leaf Huang (Editor), Mien-Chie Hung (Editor), Ernst Wagner (Editor); Academic Press; ISBN: 0123584655. However, especially contemplated transformations include delivery of the recombinant type-C lectin nucleic acid or fragment in complex with a molecule that increases the delivery specificity of the nucleic acid. For example, such molecules include substrates or substrate analogs of a receptor or transporter that causes directly or indirectly uptake of the substrate into the cell when the receptor or transporter binds the substrate (e.g., glucose transporter, transferrin receptor, or somatostatin receptor). Particularly preferred receptors and/or transporters include those that are selectively expressed in response to a particular stimulus, developmental stage, or pathophysiological stage. Therefore, it is contemplated that especially preferred cells include neoplastic cells, and the step of transforming comprises receptor-mediated uptake of the nucleic acid.

With respect to the transcription in the transformed cell, it is contemplated that the transcription is performed at least in part by the host cell. For example, where the recombinant type-C lectin nucleic acid or fragment thereof is operationally coupled to one or more control sequences, a cellular RNA polymerase may transcribe the recombinant type-C lectin nucleic acid or fragment. Alternatively, where tight regulation of transcriptional control is desirable, the recombinant type-C lectin nucleic acid may further encode a bacterial or viral RNA polymerase under control of an insect hormone promoter.

Depending on the particular design of the nucleic acid comprising the recombinant type-C lectin nucleic acid or fragment, the transcript may be an hn-RNA corresponding to the full length genomic fragment, but also a spliced and processed form of the hn-RNA, or a mRNA encoding the full length recombinant type-C lectin nucleic acid or fragment. It should therefore be appreciated that translation of such transcripts will result in a recombinant type-C lectin polypeptide or fragment thereof, and consequently the effective concentration of the type-C lectin polypeptide in the cell will increase. On the other hand, and especially where the transcript corresponds to the non-coding strand of the type-C lectin nucleic acid, it is contemplated that the transcript will hybridize in an anti-sense manner with cellular transcripts. Consequently, it is contemplated that the presence of such anti-sense transcripts will result in a reduction of the effective concentration of the type-C lectin polypeptide in the cell.

While it is generally contemplated that the binding substrate for the recombinant type-C lectin polypeptide will predominantly include the natural binding partners, it should especially be appreciated that alternative binding substrates for the type-C lectin will include conjugates with the natural binding partners. Particularly preferred conjugates may thereby include drugs that interfere with the metabolism (e.g., enzyme inhibitors), structural integrity (e.g., photodynamic substrates to promote membrane peroxidation), cell cycle (e.g., bcl-II or COD) and replication (e.g., intercalators, nucleoside analogs, etc.) of the host cell.

Further alternative uses for recombinant type-C lectin nucleic acids and fragments thereof include recombinant production of the corresponding polypeptide to obtain therapeutically useful material and protein for studies to gain better understanding of the biochemical and physiological role of the novel type-C lectin. For example, where the natural binding substrate for the novel type-C lectin is present in undesirable quantities in a subject, recombinant type-C lectins or fragments thereof may be administered to sequester excess binding substrate, and thereby help normalize the concentration of the natural binding substrate. On the other hand, where it is desirable to modulate or alter the binding characteristics of the novel type-C lectin, it is contemplated that binding studies with wild-type and mutant type-C lectin polypeptides may enhance understanding of the structure-function relationship.

(2) Diagnostic Uses

It is generally contemplated that wild-type and mutant type-C lectin polypeptides may be useful in determining the presence (and distribution where appropriate) of the natural binding substrate by coupling the recombinant type-C lectin with a detectable label (supra), and it should be appreciated that the detection of the substrate may be in vivo, ex vivo, and/or in vitro.

In an especially preferred aspect of the inventive subject matter, it is contemplated that the recombinant type-C lectin nucleic acid sequence or a fragment thereof may be employed to identify cells that produce the novel type-C lectin by hybridization methods well known in the art. Particularly preferred hybridization methods include quantitative and/or real-time PCR, membrane blots, and hybridization on a solid support (e.g., gene chip). Alternatively, detection and quantification of expression of the type-C lectin need not be limited to a cell, but may also include biopsy specimens, tissue samples, and samples from body fluids are also contemplated suitable sources for hybridization material.

Where hybridization of nucleic acids is not practicable or desirable, antibodies directed against the novel recombinant type-C lectin or fragment thereof may be employed. The generation of polyclonal and monoclonal antibodies is well known in the art, and suitable methods and protocols can be found in Antibody Production: Essential Techniques by Peter Delves (Editor); John Wiley & Son Ltd; ISBN: 0471970107 or in Monoclonal Antibodies: Principles and Practice: Production and Application of Monoclonal Antibodies in Cell Biology, Biochemistry and Immunology by James W. Goding; Academic Pr; ISBN: 0122870239.

In a particularly contemplated aspect of the inventive subject matter, antibodies may be employed to validate treatment with a drug that is imported into a target cell. In a first step, it is established that the drug enters the target cell at least in part via the novel type-C lectin, and in another step the presence of the novel type-C lectin on the cell is confirmed. Treatment with the drug is discontinued (or not started at all) when appreciable amounts on the cell surface are no more present.

Thus, specific embodiments and applications of type-C lectin nucleic acids, peptide sequences, and modifications thereof have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. An isolated polynucleotide comprising a nucleotide sequence according to SEQ ID NO:1.

2. The polynucleotide of claim 1 wherein the nucleotide sequence comprises a leader sequence domain, a transmembrane domain, and a carbohydrate recognition domain, and wherein at least one of the leader sequence domain, the transmembrane domain, and the carbohydrate recognition domain is deleted at least in part.

3. The polynucleotide of claim 2 having a sequence selected from the group consisting of SEQ ID NO:2 and SEQ ID NO:3.

4. The polynucleotide of claim 2 having a sequence according to SEQ ID NO:2.

5. The polynucleotide of claim 2 having a sequence according to SEQ ID NO:3

6. A vector comprising the nucleotide sequence of claim 1, wherein the nucleotide sequence is operationally coupled to a control sequence that is recognized by a host cell transformed with the vector.

7. A vector comprising the nucleotide sequence of claim 3, wherein the nucleotide sequence is operationally coupled to a control sequence that is recognized by a host cell transformed with the vector.

8. A host cell transformed with the vector of claim 6.

9. A host cell transformed with the vector of claim 7.

10. An isolated polypeptide having an amino acid sequence according to SEQ ID NO 4.

11. An isolated polypeptide having an amino acid sequence according to a sequence selected from the group consisting of SEQ ID NO 5 and SEQ ID NO 6.

12. The isolated polypeptide of claim 10 wherein the isolated polypeptide is coupled to a non-type-C lectin polypeptide.

13. The isolated polypeptide of claim 10 wherein the isolated polypeptide is coupled to at least a portion of an immunoglobulin.

14. The isolated polypeptide of claim 13 wherein the portion of the immunoglobulin comprises a constant part of a heavy chain of an immunoglobulin G.

15. The isolated polypeptide of claim 11 wherein the isolated polypeptide is coupled to a non-type-C lectin polypeptide.

16. The isolated polypeptide of claim 15 wherein the isolated polypeptide is coupled to at least a portion of an immunoglobulin.

17. A method of regulating the uptake of a drug into a cell, comprising:
correlating the uptake of the drug with a presence of a type-C lectin on a surface of the cell;
transforming the cell with a nucleic acid, wherein the nucleic acid encodes the type-C lectin, and wherein the nucleic acid is transcribed within the cell to produce a transcript; and
wherein the transcript at least indirectly alters an amount of the type-C lectin on the surface of the cell.

18. The method of claim 17 wherein the cell is a neoplastic cell, and the step of transforming comprises receptor-mediated uptake of the nucleic acid.

19. The method of claim 17 wherein the transcript is translated, thereby increasing the amount of the type-C lectin on the surface of the cell.

20. The method of claim 17 wherein the transcript hybridizes with a cellular nucleic acid encoding the type-C lectin, thereby decreasing the amount of the type-C lectin on the surface of the cell.


SEQUENCE LISTING (6 sequences)

SEQ ID NO: 1 - DNA, 648 bp, Homo sapiens
  sig_peptide (1)..(118)
atggggctagaaaaacctcaaagtaaactggaaggaggcatgcatccccagctgataccttcggttattgctgtagttttcatcttacttctcggtgtctgttttattgcaagttgtttggtgactcatcacaacttttcacgctgtaagagaggcacaggagtgcacaagttagagcaccatgcaaagctcaaatgcatcaaagagaaatcagaactgaaaagtgctgaagggagcacctgggaactgttgtcctatgactggagagccttccagtccaactgctattttcctcttactgacaacaagacgtgggctgagagtgaaaggaactgttcagggatgggggcccatctgatgacatcagcacggaagctgagcagaactttattattcagtttctggatagacggctttcctatttccttggacttagagatgagaatgccaaagggtcagtggcgttgggtggaccagacgccatttaacccacgcagagtattctggcataagaatgaacccgacaactctcagggagaaaactgtgttgttcttgtttataaccaagataaatgggcctggaatgatgttccttgtaactttgaagcaagtaggatttgtaaaatacctggaacaagattgaactag

SEQ ID NO: 2 - DNA, 63 bp, Homo sapiens
  misc_structure (1)..(63): Transmembrane domain
tcggttattgctgtagttttcatcttacttctcggtgtctgttttattgcaagttgtttggtg

SEQ ID NO: 3 - DNA, 375 bp, Homo sapiens
  misc_feature (1)..(375): Carbohydrate Binding Domain
ttgtcctatgactggagagccttccagtccaactgctattttcctcttactgacaacaagacgtgggctgagagtgaaaggaactgttcagggatgggggcccatctgatgacatcagcacggaagctgagcagaactttattattcagtttctggatagacggctttcctatttccttggacttagagatgagaatgccaaagggtcagtggcgttgggtggaccagacgccatttaacccacgcagagtattctggcataagaatgaacccgacaactctcagggagaaaactgtgttgttcttgtttataaccaagataaatgggcctggaatgatgttccttgtaactttgaagcaagtaggatttgtaaa

SEQ ID NO: 4 - protein, 215 aa, Homo sapiens
  SIGNAL (1)..(39)
MGLEKPQSKLEGGMHPQLIPSVIAVVFILLLGVCFIASCLVTHHNFSRCKRGTGVHKLEHHAKLKCIKEKSELKSAEGSTWELLSYDWRAFQSNCYFPLTDNKTWAESERNCSGMGAHLMTSARKLSRTLLFSFWIDGFPISLDLEMRMPKGQWRWVDQTPFNPRRVFWHKNEPDNSQGENCVVLVYNQDKWAWNDVPCNFEASRICKIPGTRLN

SEQ ID NO: 5 - protein, 21 aa, Homo sapiens
  TRANSMEM (1)..(21)
SVIAVVFILLLGVCFIASCLV

SEQ ID NO: 6 - protein, 125 aa, Homo sapiens
  BINDING (1)..(125): Carbohydrate Binding Domain
LSYDWRAFQSNCYFPLTDNKTWAESERNCSGMGAHLMTSARKLSRTLLFSFWIDGFPISLDLEMRMPKGQWRWVDQTPFNPRRVFWHKNEPDNSQGENCVVLVYNQDKWAWNDVPCNFEASRICK